(19) United States
(12) Patent Application Publication
(10) Pub. No.: US 2022/0268754 A1
(43) Pub. Date: Aug. 25, 2022

(54) EARLY DETECTION OF E. COLI AND TOTAL COLIFORM USING AN AUTOMATED, COLORIMETRIC AND FLUOROMETRIC FIBER OPTICS-BASED DEVICE

(71) Applicant: THE REGENTS OF THE UNIVERSITY OF CALIFORNIA, Los Angeles, CA (US)

(72) Inventors: Aydogan Ozcan, Los Angeles, CA (US); Kevin de Haan, Los Angeles, CA (US); Hatice Ceylan Koydemir, Los Angeles, CA (US); Sabiha Tok, Los Angeles, CA (US); Derek Tseng, Buena Park, CA (US)

(73) Assignee: THE REGENTS OF THE UNIVERSITY OF CALIFORNIA, Los Angeles, CA (US)

(21) Appl. No.: 17/627,400

(22) PCT Filed: Jul. 29, 2020
(86) PCT No.: PCT/US20/44032

§ 371 Date: Jan. 14, 2022

Related U.S. Application Data
(60) Provisional application No. 62/880,029, filed on Jul. 29, 2019.

(51) Int. Cl. G01N 33/18; G01N 21/64; G02B 6/04; F21V 8/00; G01N 21/78

(52) U.S. Cl. CPC G01N 33/1826; G01N 21/6452; G02B 6/04; G02B 6/0008; G01N 21/6456; G01N 21/78; G01N 2021/6484; G01N 2021/7786

(57) ABSTRACT

A system for detecting the presence of E. coli and total coliform in a water sample includes a sample holder that holds smaller, divided volumes of the sample and a testing reagent. A plurality of light sources are disposed above sample holder. The divided sample volumes are are illuminated with first and second light sources emitting light at different wavelengths. A bundle of optical fibers is provided with having an input end located adjacent to the divided sample volumes and is configured to receive light passing through the sample volumes. Light is output from the bundle of optical fibers and is captured with a camera. Image processing software is provided and is configured to calculate a light intensity in first and second wavelength channels at different times and outputs a positive/negative indication for E. coli and total coliform for the water sample.

### RELATED APPLICATION

This Application claims priority to U.S. Provisional Patent Application No. 62/880,029 filed on Jul. 29, 2019, which is hereby incorporated by reference in its entirety. Priority is claimed pursuant to 35 U.S.C. § 119 and any other applicable statute.

### TECHNICAL FIELD

The technical field generally relates to devices and methods used to detect and quantify the number of bacteria in a water sample. More specifically, the technical field relates to a small, portable device that can automatically detect the presence of both Escherichia coli (E. coli) and total coliform in drinking water within ˜16 hours, down to a level of one colony-forming unit (CFU) per 100 mL.

### STATEMENT REGARDING FEDERALLY SPONSORED RESEARCH AND DEVELOPMENT

This invention was made with government support under Grant Number W911NF-17-1-0161, awarded by the U.S. Army, Army Research Office. The government has certain rights in the invention.

### BACKGROUND

The World Economic Forum's “The Global Risks Report 2019” states that water crises have been one of the top 5 global risks in terms of impact, and the top societal risk for the past 5 years. According to the World Health Organization (WHO), 785 million people lack safe drinking water, and at least 2 billion people use water sources contaminated with feces. Lack of access to safe, contaminant-free water, severely threaten public health due to waterborne illnesses. It is estimated that 1 million people die every year due to water, sanitation or hygiene related problems, and every 2 minutes a child dies due to poor quality water. Therefore, effective monitoring of water quality is urgently needed to prevent waterborne diseases, improve public health, and save lives.

Solving these problems is no simple task. Water can contain hundreds of different microorganisms, making the analysis of all possible pathogenic microorganisms very challenging. However, the presence of E. coli and total coliform in a water sample is widely accepted as evidence for contamination of a water supply. Total coliform bacteria are commonly found in the environment. Therefore, the presence of total coliform in water samples is an indicator of contamination from the surrounding environment. While the coliform bacteria do not necessarily cause disease, their presence can indicate that other pathogens may potentially exist within the water sample. On the other hand, E. coli is a member of the fecal coliform group, which exists in the intestines and feces of human and other warm-blooded animals. Therefore, the presence of fecal coliform, more specifically E. coli, indicates the presence of disease-causing pathogens. In practice, monitoring only E. coli and total coliform contamination is sufficient to analyze water quality for health-related risks. According to the United States Environmental Protection Agency (EPA) in order to determine whether or not a water source is safe for drinking, the sensitivity of the measurement technique must be at least 1 CFU/100 mL.

There are several EPA-approved methods used to monitor water quality which employ conventional microbiological techniques such as multiple tube fermentation and membrane filtration. However, these microbiological methods have some limitations, such as a long total analysis time, interference from non-coliform bacteria, limited detection of slow-growing or stressed coliform, viable but non-culturable (VBNC) bacteria, and requiring transportation to central lab facilities with trained professionals. There are other emerging methods such as immunological assays and polymerase chain reaction (PCR) based methods which in general provide faster detection. However, these methods require relatively complex procedures and trained specialists. Additionally, these methods do not allow on-site water quality monitoring. Instead, water samples need to be transported to central lab facilities, resulting in an additional delay. Water quality can also be monitored by optical, electrochemical, piezoelectric or plasmonic biosensors. However, such biosensor technologies typically lack sensitivity and/or are constrained to very small sample volumes, in addition to requiring complex and expensive benchtop equipment to operate.

One of the EPA-approved methods for E. coli and total coliform detection is Colilert® (IDEXX Laboratories, Inc., Westbrook, Me.). This is one of the most widely used technique, and is an enzymatic method which uses Defined Substrate Technology (DST) to simultaneously detect E. coli and total coliform in drinking water. Within the Colilert® reagent there are two substrates: o-nitrophenyl-β-D-galactopyranoside (ONPG) and 4-methylumbelliferyl-β-D-glucuronide (MUG), which are metabolized by the coliform enzyme β-galactosidase and E. coli enzyme β-glucuronidase respectively. When total coliform bacteria are present in the water sample, they use the β-galactosidase to metabolize ONPG, which releases o-nitrophenol and changes the sample from being colorless to yellow. E. coli use β-glucuronidase to metabolize MUG and release 4-methylumbelliferone (4-MU), which is a fluorescent molecule and emits blue light when excited by ultraviolet (UV) light. This method is sensitive and can be used to detect concentrations as low as 1 CFU/100 mL, and quantification is available using a most probable number (MPN) table or software. However, when used to quantify coliform bacteria concentration in water samples, the Colilert® method has drawbacks as well. Notably, the total process takes 24 to 28 hours, and similar to those listed above, it is not an on-site method (i.e., the samples must be transported to a lab with trained personnel and special equipment). In case of fecal contaminated water sources, it is crucial to detect the presence of the bacteria as early as possible to prevent illness. To achieve this, a sensitive, portable and cost-effective water quality sensor or device which can be operated by non-specialists is urgently needed.

### SUMMARY

In one embodiment, a cost-effective and highly sensitive water quality monitoring device (or sensor) is provided which can perform automatic early detection of both E. coli and total coliform using the Colilert® reagent, mixed with the sample water under test, which is then placed inside a custom-designed 40-well plate to be automatically imaged all in parallel using fiber optic cables. The device, in one specific implantation, weighs 1.66 kg and can automatically detect 1 CFU/100 mL in less than 16 h, which allows the sample to be processed using limited laboratory equipment and without requiring specialized personnel. At higher concentrations of E. coli and/or total coliform the automated detection time can be further decreased.

In one embodiment, a system for detecting the presence of E. coli and total coliform in a water sample includes a portable device that contains a sample holder therein configured to hold a plurality of smaller volumes of the sample and a testing reagent therein. These may include vials, tubes, wells, or the like which are optically transparent. A plurality of light sources are disposed above the plurality of smaller volumes of the sample, wherein each of the plurality of smaller volumes of the sample within the sample holder is illuminated with first and second light sources. These may include an array of LEDs with two LEDs (blue and ultraviolet (UV) emitters) situated above each smaller volume of sample. A bundle of optical fibers is provided having an input end and an output end, the input end of the bundle comprising a plurality of optical fibers located adjacent to the plurality of smaller volumes of the sample and configured to receive light passing through the plurality of smaller volumes of the sample from the first and second light sources. A long-pass filter may be interposed between the sample holder and the input end of the bundle of optical fibers. A lens is disposed in the device and configured to receive light emitted from the output end of the bundle of optical fibers. A camera is included in the device/sensor and is configured to capture images of the light passing through the lens from the bundle of optical fibers for the plurality of smaller volumes of the sample. The system includes image processing software configured to calculate a light intensity in first and second channels corresponding to the first and second light sources at different times, wherein the image processing software outputs a positive/negative indication for E. coli and total coliform for the water sample. The change in intensity over time (over successive measurements) is used to make the positive/negative determination. The output may also include the concentration (or concentration range of E. coli and total coliform). The image processing software may be run using a separate computing device (e.g., laptop, PC, server, tablet, mobile phone, etc.) that executes the image processing software. For example, image files may be transferred or offloaded to this other computing device for processing. Of course, the computing device may also be integrated into the portable device itself in other embodiments. In this embodiment, there would be no need for the transfer or offloading of the images as they would be processed directly by the device/sensor.

In one embodiment, a system for detecting the presence of E. coli and total coliform in a water sample is disclosed. The system includes a sample holder configured to hold a plurality of smaller volumes of the sample and a testing reagent therein. A plurality of light sources are disposed above the plurality of smaller volumes of the sample, wherein each of the plurality of smaller volumes of the sample within the sample holder are illuminated with first and second light sources of the plurality of light sources emitting light at different wavelengths. The system includes a bundle of optical fibers having an input end and an output end, the input end of the bundle comprising a plurality of optical fibers located adjacent to the plurality of smaller volumes of the sample and configured to receive light passing through the plurality of smaller volumes of the sample from the first and second light sources. A lens is provided and is configured to receive light emitted from the output end of the bundle of optical fibers. The lens focuses the light from the output end of the bundle of optical fibers to a camera configured to capture images of the light passing through the lens from the bundle of optical fibers. The system includes computing device that runs image processing software that configured to calculate a light intensity in a first wavelength channel and a second wavelength channel at different times, wherein the image processing software outputs a positive/negative indication for E. coli and total coliform for the water sample. The image processing software may also output a concentration or concentration range of E. coli and total coliform for the water sample. The computing system may be integrated as part of the testing device or a separate computing device and image files are transferred from the device to the separate computing device.

In another embodiment, a method for detecting the presence of E. coli and total coliform in a water sample includes mixing the water sample and a testing reagent; dividing the mixture into a plurality of smaller volumes of the water sample and the testing reagent; loading the plurality of smaller volumes of the water sample and the testing reagent into the sample holder and inserted into the testing device. The testing device then periodically illuminates the plurality of smaller volumes of the water sample and the testing reagent with light from the first light source and capturing images of the light passing through the lens from the bundle of optical fibers associated with the plurality of smaller volumes of the water sample. The testing device also periodically illuminates the plurality of smaller volumes of the water sample and the testing reagent with light from the second light source and capturing images of the light passing through the lens from the bundle of optical fibers associated with the plurality of smaller volumes of the water sample. The images captured at the camera are then processed with image processing software configured to calculate a light intensity in first and second channels at the periodic times, wherein the image processing software outputs a positive/negative indication for E. coli and total coliform for the water sample based on a change in light intensity over time. The image processing software may also output a concentration or concentration range of E. coli and total coliform for the water sample.

### BRIEF DESCRIPTION OF THE DRAWINGS

FIGS. 1A-1B schematically illustrate the components of the E. coli and total coliform detection device according to one embodiment. FIG. 1A illustrates a detailed illustration of the key components. FIG. 1B illustrates the working principle for a single well (e.g., divided sample holder). A pair of blue and UV LEDs, controlled by the microcontroller or processor (e.g., Raspberry Pi), are used to illuminate each well of the 40-well plate. The blue light passing through the well and the fluorescence emitted by the sample in response to UV excitation are long-pass filtered. Then the light passing through each well is collected by the bundle of optical fibers and mapped onto the camera.

FIG. 1C schematically illustrates the system for detecting the presence of E. coli and total coliform in a water sample according to one embodiment.

FIGS. 2A-2C: E. coli and coliform concentrations detected by the device up to 200 CFU/100 mL, compared to the plate count method used as the gold standard, ground truth. The x-axis represents the number of bacteria measured by the plate count method and the y-axis (left) represents the number of positive wells detected by the device. The right side of the y-axis shows the theoretical number of positive wells. FIG. 2A shows results for E. coli measurements, FIG. 2B shows results for E. aerogenes measurements, FIG. 2C shows results for C. freundii measurements.

FIGS. 3A-3C illustrate graphs showing the time that the device takes until the first detection of a positive signal in the sample being measured. FIG. 3A shows E. coli measurements, FIG. 3B shows E. aerogenes measurements, FIG. 3C shows C. freundii measurements.

FIGS. 4A-4D illustrate how the detection device is capable of performing measurements faster than a manual count. The device detects the slow-growing coliforms in less than 24 hours, while the visual inspection cannot. FIG. 4A is a photograph of the wells after 24 hours of incubation. FIG. 4B is a photograph of the wells after 25 and 28 hours of incubation, where two (25 h) and four (28 h) additional wells have become positive. FIG. 4C is a plot of the absorption channel intensity over time measured by the device. FIG. 4D is a table showing the time (hours) at which the device was able to detect each positive well.

FIGS. 5A-5C illustrate graphs demonstrating the detection times for different E. coli concentrations. FIG. 5A shows the time until the first detection of E. coli as a function of the bacteria concentration. FIG. 5B illustrates the measured intensity of the fluorescence channel, and FIG. 5C illustrates the measured intensity of the absorption channel intensity measured by the device over time, for increasing concentrations of E. coli.

FIGS. 6A-6B illustrate preparation of the samples used to test the device with FIG. 6A showing the workflow used to prepare the 100 mL sample for testing. FIG. 6B illustrates the workflow used to prepare the 5 agar plates used as the gold standard.

FIG. 6C illustrates visualization of the results for the device, showing the positive wells (left), a plot of intensity over time measured by the device (middle), and a visualization of the plate count (right). For the fluorescence channel, a flat line indicates a negative well, and a spike indicates the presence of E. coli. For the absorption channel, a flat line indicates a negative well, and a drop in the transmission intensity indicates the presence of coliform bacteria.

FIGS. 7A-7D illustrate photographs and results of negative control experiments (n=3). For the negative tests, there is no bacteria growing either on the plates or within any of the wells. The time series for both the fluorescence and absorption (e.g., blue) channels are flat, and the device does not classify any of the wells as positive. FIG. 7A shows photographs of the agar plates which do not have any bacteria growing on them after 24 hours. FIG. 7B shows photographs of the 40-well plates after 24 hours of incubation. FIGS. 7C and 7D illustrate the normalized intensity over time for the fibers in each well (FIG. 7C—fluorescence channel; FIG. 7D—absorption channel).

FIGS. 8A-8C illustrate an example of the sample preparation steps for a wide range of E. coli concentrations. FIG. 8A illustrates adding the Colilert® reagent into the water sample and loading the desired volume to the vials of the 40-well plate. FIG. 8B illustrates schematically the 10-fold serial dilution. FIG. 8C shows the preparation of the agar plates (n=3), where the last four concentrations are used as the gold standard to validate the device.

FIG. 9A illustrates sample preparation steps of adding Colilert® reagent into 100 mL water sample and loading it into the 40-well plate. The well plate is then put inside the device and incubated at 35° C.

FIG. 9B illustrates an example of the automated output of the device which reports the presence of both E. coli and total coliform bacteria.

### DETAILED DESCRIPTION OF THE ILLUSTRATED EMBODIMENTS

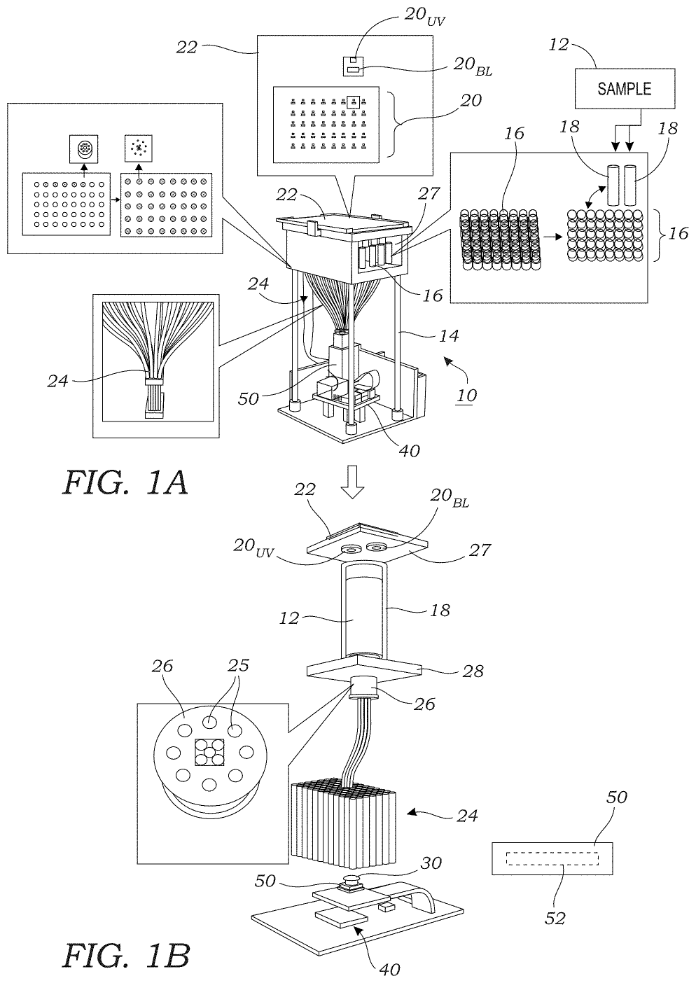

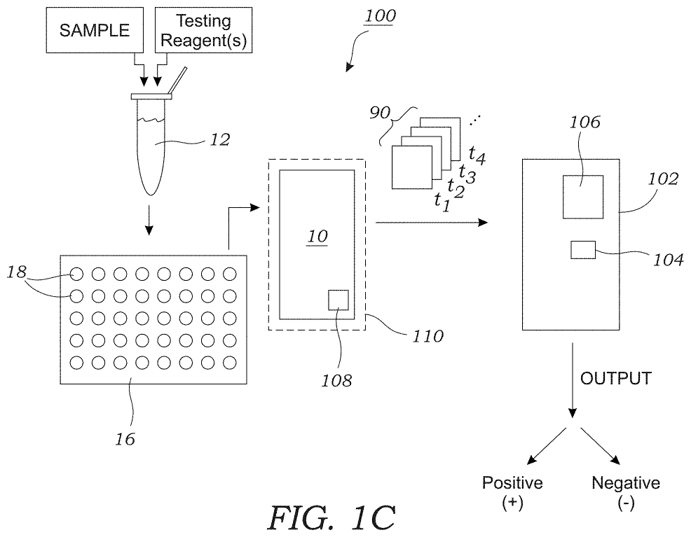

FIGS. 1A-1C illustrates a device 10 for detecting the presence of E. coli and total coliform in a water sample 12. While water is the preferred fluid to be tested, other liquid fluids may also be tested in a similar manner. FIG. 1C illustrates a system 100 that includes the device 10. With reference to FIGS. 1A and 1B, the device 10 includes a housing 14 that contains the various components of the device 10. The device 10 is small and portable, weight less than 2 kg in one embodiment. The small portable device 10 may be located in an incubator 110 (illustrated in FIG. 1C) or the like during the testing process to maintain the desired temperature and environmental conditions for bacterial growth. The housing 14 also prevents ambient light from interfering with the imaging operation although the incubator 110 may also be used to prevent ambient light from interfering with imaging operations as discussed herein.

The device 10 includes a sample holder 16 configured to hold a plurality of smaller volumes of the water sample 12 and a testing reagent therein. That is to say, in one embodiment, a single water sample 12 is divided into smaller volume samples which are loaded into their own vessels or containers 18 (e.g., vials, tubes, receptacle, well, or the like). In one embodiment, the individual vessels or containers 18 are detachable vials that are mounted in a sample holder 16 that receives the vessels or containers 18. For example, the sample holder 16 may be manufactured using three-dimensional printing with receptacles that receive each vessel or container 18. In the illustrated embodiment, the sample holder 16 can hold forty (40) vessels or containers 18 containing the water sample to be tested. It should be appreciated, however, that more or fewer vessels or containers 18 may be used in conjunction with the device 10. The sample holder 16 may be made from an optically transparent material or have apertures or holes that allow for the transmission of light therethrough. The sample holder 16 with the vessel or containers 18 (loaded with fluid) contained therein may be inserted into the device 10. The vessels or container 18 may be covered with a UV transmissible film, cover, or cap to seal the liquid contained therein.

A plurality of light sources 20 (e.g., LEDs, laser diode, or the like) are disposed in the housing 14 above the array of vessels or containers 18 contained sample holder 16 that is loaded into the device. An optional diffuser (not shown) may be used to diffuse the light emitted from the plurality of light sources 20. The plurality of light sources 20 thus are used to illuminate the plurality of smaller volumes of the water sample 12. In one embodiment, the plurality of light sources 20 includes at least one light source 20 that emits light at a first wavelength and at least one light source 20 that emits light at a second wavelength. For example, as seen in FIG. 1A, in this embodiment, there are a plurality of light sources 20 arrayed on a printed circuit board (PCB) 22. Driver circuitry (not shown) may also be located in the PCB 22. In this embodiment, the plurality of light sources 20 include a plurality of LEDs that emit ultraviolet (UV) light (i.e., LED 20_{UV} in FIG. 1A) and a plurality of LEDs that emit blue light (i.e., LED 20_{BL} in FIG. 1A). As seen in FIG. 1A, there are a total of eighty (80) LEDs with forty LEDs being blue LEDs 20_{BL} and forty LEDs being UV LEDs 20_{UV}. Of course, different numbers of LEDs or light sources may be used. At least one UV light source 20_{UV} and at least one blue light source 20_{BL} is needed to illuminate the smaller volumes of water sample 12. Of course, additional light sources 20 will more evenly illuminate the array of vessels or containers 18. Thus, in one embodiment, each vessel or container 18 is associated with a single UV light source 20_{UV} and a single blue light source 20_{BL}.

As explained herein, the UV light source(s) 20_{UV} and the blue light source(s) 20_{BL} illuminate the plurality of smaller volumes separately. This means that when the UV light source(s) 20_{UV} are “ON” the blue light source(s) 20_{BL} are “OFF.” Conversely when the blue light source(s) 20_{BL} are “ON” the UV light source(s) 20_{UV} are “OFF.” Driver circuitry and/or a microcontroller 40 or other processor may be used to power the plurality of light sources 20. The microcontroller 40 or other processor may also be used to control the timing or sequencing of the UV light source(s) 20_{UV} and the blue light source(s) 20_{BL}. The microcontroller 40 or other processor may also control the camera 50 and acquisition and optional transfer of images 90. In some embodiments, the image processing software 106 may also be executed by the microcontroller 40 or other processor. As explained herein, images 90 of transmitted and/or fluorescent light passing through or emitting from the vessels or containers 18 are captured with a camera 50. The captured images 90 enable the system 100 to determine the intensity of the measured light at various time intervals which are then used to determine whether the particular vessel or container 18 is positive (+) or negative (−).

The device 10 includes a bundle of optical fibers 24 having an input end and an output end. The input end of the bundle of optical fibers 24 are located adjacent to the vessels or containers 18 containing the smaller volumes of the water sample 12 and are configured to receive light from the respective samples contained in the vessels or containers 18 in response to illumination from the plurality of light sources 20. The light that is received by the input end of the bundle of optical fibers 24 is either light that is transmitted through the water sample(s) 12 within the vessels or containers 18 or fluorescent light that is generated within the water sample(s) 12 within the vessels or containers 18.

In one embodiment, as best seen in FIG. 1B, a plurality of optical fibers from the bundle of optical fibers 24 are located in a header 26 that is used to collect light from a single vessel or container 18. For example, a header 26 is illustrated that contains thirteen (13) optical fibers 25 from the bundle of optical fibers 24. The header 26 may be disposed adjacent to the bottom of the vessel or container 18 so that light that is transmitted (or generated therein in the case of emitted fluorescence) is captured by these thirteen (13) optical fibers 25 from the bundle of optical fibers 24. The light is then transmitted and captured by the camera 50.

For the samples 12 that fluoresce, the fluorescent light is filtered using, for example, a long-pass filter 28 that is interposed between the vessels or containers 18 that contain the smaller volumes of sample 12 and the input end of the bundle of optical fibers 24. The long-pass filter 28 does not affect the blue light emitted by the blue light source(s) 20_{BL}. A lens 30 is disposed adjacent to the output end of the bundle of optical fibers 24 and is configured to receive light emitted from the output end of the bundle of optical fibers 24 and focus/direct the light onto the camera 50. The camera 50 is configured to capture images 90 of the light passing through the lens 30 from the bundle of optical fibers 24 for each of the vessels or containers 18 containing the plurality of smaller volumes of the sample. The images 90 may include spots or regions of light intensity corresponding to each fiber 25 of the bundle of optical fibers 24. The individual optical fibers 25 that make up the bundle of optical fibers 24 may be mapped to specific pixels within the image 90 so that intensity values can be associated with a particular vessel or container 18. The camera 50 illustrated in the device 10 used to generate the experimental results was a Raspberry Pi camera but it should be appreciated that other cameras 50 may be used. The camera 50 includes an image sensor 52 (FIG. 1C) that is used to capture the actual image 90. The image sensor 52 may include a standard CMOS image sensor 50 known to those skilled in the art.

In a preferred embodiment, the light intensity at the first and second channels (i.e., fluorescence channel and absorption channel) is monitored at periodic intervals (e.g., 15 minutes) and the change in intensity is used to determine the positive/negative indication for E. coli and total coliform. In one embodiment, so long as one of the plurality of smaller volumes contained in the vessel or container 18 undergoes a change in intensity that meets an established threshold value, the sample may be classified as “positive.” The testing may continue for several hours or longer than a day while the plurality of smaller samples are incubated and periodically imaged.

With reference to FIG. 1C, the system 100 includes a computing device 102 that includes one or more processors 104 therein that are used to execute image processing software 106 that is configured to calculate a light intensity in the fluorescence and absorption channels. The image processing software 106 may include MATLAB® (The MathWorks, Inc.) or other commercially known software packages. First, the exact location of the center of each optical fiber of the bundle of optical fibers 24 is located by using the first image 90 illuminated with the blue light source(s) 20_{BL}. These optical fibers are then associated or mapped with the different vessels or containers 18 using a pre-set manually labelled groupings (done previously to associate or map particular fibers to a particular vessel or container 18). To account for minor shifts, later images 90 are then registered to the initial image using cross correlation.

After the above pre-processing operations, the respective fiber intensities in each image 90 are then measured by summing the intensities of all the pixels within a radius of the fiber's center (e.g., 14-pixel radius used herein). Because there may be some crosstalk between different vessels or containers 18 in the fluorescence channel, the intensity values from the fibers associated with each vessel or container 18 are reduced according to the intensity of those around them. This is a normalization process di done by multiplying the average intensity of the thirteen (13) optical fibers under each vessel or container 18 by an empirically determined constant and dividing it by the square of the distance between the vessels or containers 18.

The normalized intensities of the fibers 25 in each vessel or container 18 are averaged for the final classification. The absorption channel, which is used for the detection of total coliform, is in one embodiment, classified as positive when the intensity drops by 5% over ten (10) successive images 90. The fluorescence channel is classified using a manually-chosen threshold, where the vessel or container 18 is marked as positive if the intensity increases more than 20% of the value after the first 75 min, indicating that E. coli is present in the sample 12. In both cases, the first five (5) time points (i.e., the first hour after loading the sample inside the incubator 110) are ignored as there are significant fluctuations in the signal intensities due to the liquid in the vessels or containers 18 slowly warming to the temperature of the incubator 110.

As seen in FIG. 1C, the image processing software 106 outputs a positive (+)/negative (−) indication for E. coli and total coliform for the water sample 12. The output may also include the concentration (or concentration range of E. coli and total coliform). This may be based on the number of vessels or containers 18 that are positive at a particular point in time. See Eq. 2 herein. In one embodiment, the sample 12 is presumed to be negative until a single vessel or container 18 is established to be positive (+) for E. coli and/or total coliform. For example, for E. coli the particular test may deem the entire sample 12 positive when only a single vessel or container 18 is deemed positive. Alternatively, the entire sample 12 may be deemed positive when a plurality of vessels or containers 18 is deemed positive. The cutoff for a positive or negative sample may depend on the number of single vessel or container 18 as well as regulatory or legal cut-offs for positive or negative samples which may vary with different jurisdictions. In some embodiments, the computing device 102 may be integrated into the device 10. For example, the software may be run using the microcontroller 40 or other on-board processors in some embodiments. Alternatively, the images 90 may be transferred or offloaded to a local or remote computer that contains the image processing software 106. The device is powered by a power source 108 (FIG. 1C) such as a battery that is used to power the plurality of light sources 20, camera 50, and microcontroller 40 or other electronics. The device 10 may be provided with its own incubator 110 or, alternatively, existing laboratory incubators 110 may be used with the device 10 being inserted therein.

Experimental

Materials

The three different types of bacteria, Escherichia coli (E. coli) (ATCC® 25922™) Klebsiella aerogenes (K. aerogenes or Enterobacter aerogenes (E. aerogenes)) (ATCC® 49701™), and Citrobacter freundii (C. freundii) (ATCC® 43864™) were obtained from American Type Culture Collection, Manassas, Va., USA. Tryptic Soy Agar (TSA) (BD Difco) and Nutrient Agar (NA) (BD Difco) were obtained from Fisher Scientific, CA, USA. Colilert® reagent for 100 mL water sample (Colilert® snap packs) and Colilert® sterile vessels were obtained from IDEXX Laboratories, Inc., Westbrook, USA. Multimode optical fibers 25 (FT400UMT) were used for the bundle of optical fibers 24, the ground glass diffuser (DG100X100-220 N-BK7) and a plano-convex lens 30 (LA1027-A) were purchased from Thorlabs, Newton, N.J., USA. The ultraviolet light emitting diodes (UV LEDs 20_{UV}) (VLMU1610-365-135CT-ND) and drivers (296-31235-ND) were obtained from Digi-Key Electronics, Thief River Falls, Minn., USA. The blue LEDs 20_{BL} (749-SM1206UV-400-IL) were obtained from Mouser Electronics, Mansfield, Tex., USA. The Raspberry Pi Board (Raspberry Pi 3 Model B) 40 was purchased from Newark element 14, Chicago, Ill., USA, and the camera 50 (Raspberry Pi Camera Module V2-8 Megapixel, 1080p) was purchased from Raspberry Pi Foundation, UK. Rechargeable batteries were obtained from EBL, LA, USA. The Plexiglas MC acrylic sheet UF-5 was purchased from Altuglas International Arkema Inc., Philadelphia, Penn., USA. The glass beads (ColiRollers Sterile Plating Beads (Novagen)) were purchased from MilliporeSigma, Burlington, Mass., USA. The sealing film (ThermalSeal RTS™, Sterile) was obtained from Sigma-Aldrich, Burlington, Mass., USA. Reagent Grade Water (Nerl, Reagent Water (CLRW) and USP/NF Purified Water) and Petri dishes (100 mm×15 mm, Sterile) were obtained from Fisher Scientific, CA, USA. Microcentrifuge tubes (Micrewtube, 2 mL, o-ring seal screw cap, sterile) were obtained from Thomas Scientific, Swedesboro, N.J., USA. Glass shell vials (Kimble) were obtained from Carolina Biological Supply Company, Burlington, N.C., USA. The microbiological incubator (Isotemp) and autoclave (SterilElite) were obtained from Fisher Scientific, CA, USA. The incubator (Heracell VIOS CO₂) was obtained from Thermo Scientific, Waltham, Mass., USA.

Culture Based Assays and Sample Preparation

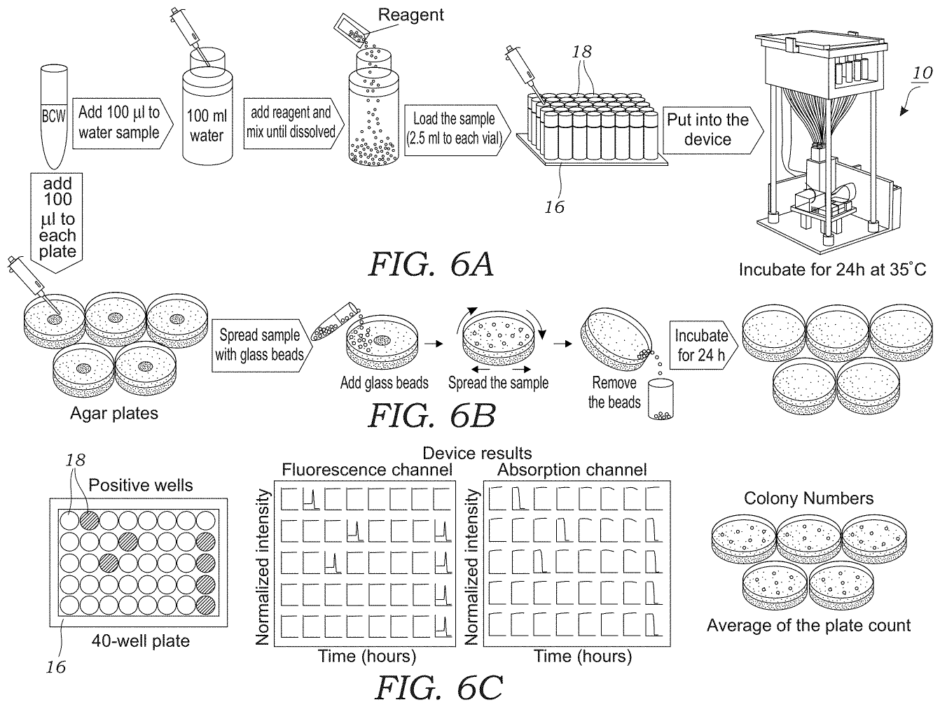

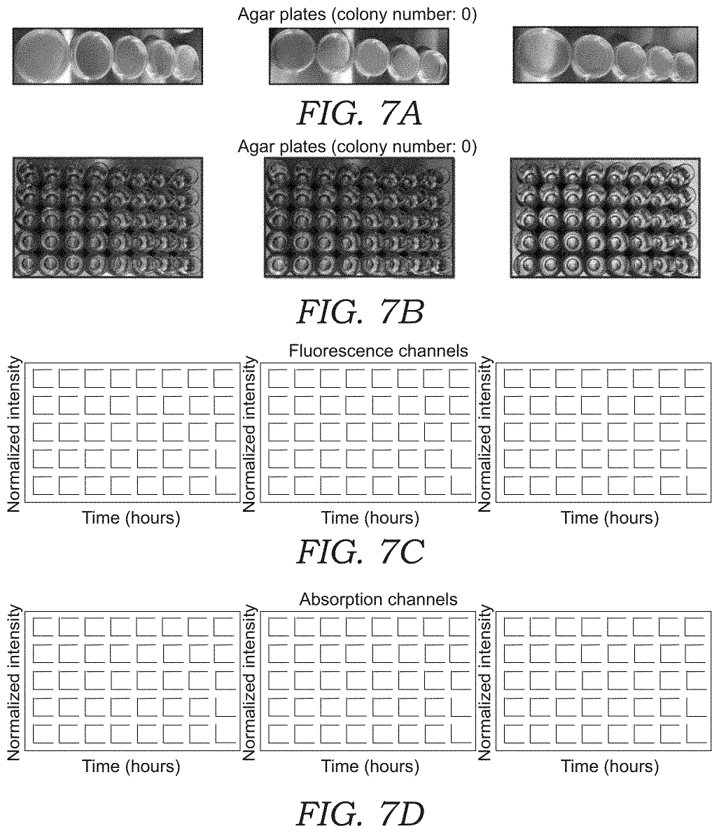

Pure cultures of E. coli (ATCC 25922) and E. aerogenes (ATCC 49701) were grown on Tryptic Soy Agar (TSA) for 24 h in a microbiological incubator at 37° C. and 35° C., respectively. C. freundii (ATCC 43864) was cultured on Nutrient Agar (NA) at 37° C. in an incubator. TSA and NA plates were prepared according to the manufacturer's specifications. Following this, 20 mL was poured into each 100 mm diameter plate and used or stored at 4° C. until use. The agar plates are used to culture the bacteria and to perform quantitative measurements of the bacteria concentration for comparison to the presented method. This plate count, in which bacteria were grown on agar plates and counted, was used as the “gold standard” for determining the concentration added to the device during testing. Bacteria from an overnight culture were resuspended in 1 mL sterile reagent grade water and serially diluted (10-fold) as required and 100 μl of bacteria contaminated water (BCW) sample was added to each agar plate (n=5). Samples were spread onto the agar surface with sterile glass beads using the Copacabana method and the plates were incubated for 24 h. After overnight incubation, individual colonies of bacteria on the agar plates were counted and averaged (see FIGS. 6A-6C). In addition to the bacteria samples, negative control experiments were performed using the same procedures without any addition of bacteria (see FIGS. 7A-7D).

To prepare the samples 12 used to validate the performance of the device 10, 100 μL of the same BCW sample described above was added to 100 mL of sterile reagent grade water and mixed with the Colilert® reagent until dissolved. 2.5 mL of the contaminated water sample 12 was then added to each sterile glass vial 18 within the 40-well plate 16. The filled vials 18 were then sealed with sterile, non-fluorescent, UV-transmitting sealing film, put into the device 10 and incubated for 24 h at 35° C. inside the incubator 110. Following the incubation, the concentration of bacteria which was determined with the plate count (n=5) was compared with the automated counting results of the device 10 (see FIGS. 6A-6C). Negative control experiments were performed using the same procedure without bacteria added to the initial sample.

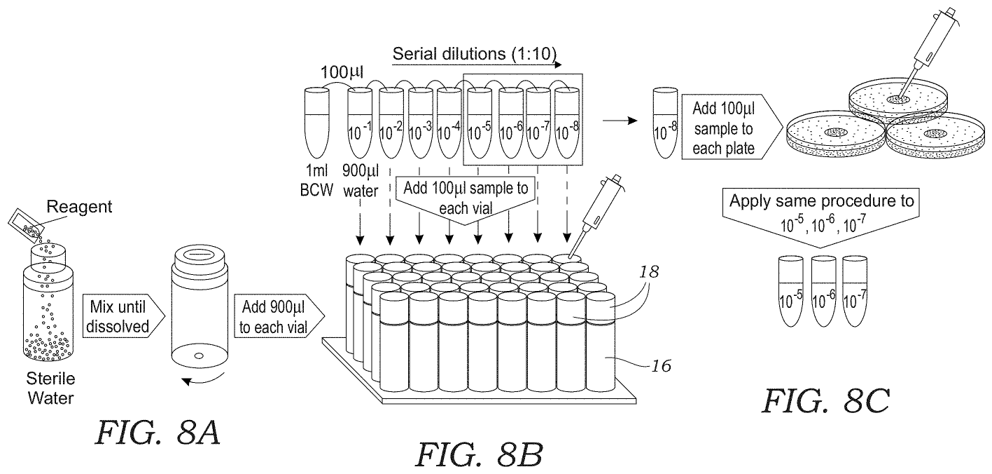

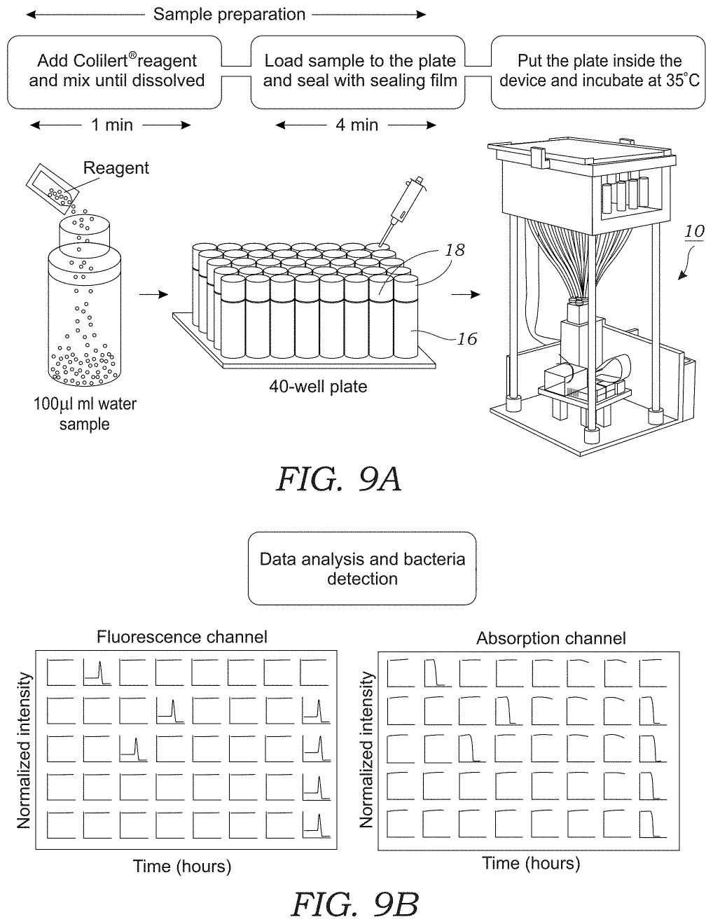

To test the performance of the device 10 at higher concentrations of bacteria, a modified version of the above test was used. The sample preparation steps for these tests can be visualized in FIGS. 8A-8C. For these tests, eight different E. coli concentrations were prepared with 10-fold serial dilution between each. The Colilert® reagent was added to 100 mL sterile reagent grade water and 900 μl of this sample was in turn added to each vial 18 of the 40-well plate 16. 100 μl of each E. coli concentration was added to five (5) vials 18, and similar to the procedures outlined above, the 40-well plates 16 were sealed and incubated for 24 h at 35° C. To quantitatively determine the precise bacteria concentrations used for these tests, the plate count method (n=3) was applied following the procedure described herein. Since higher concentrations are too numerous to count (TNTC), the plate count method is applied to only the four lowest concentrations.

Device Design

To use the device 10 a water sample 12 of interest is split evenly into forty (40) disposable glass vials 18 which are held by a custom 3D-printed 40-well plate sample holder 16. There are two LEDs 20 (one UV, one blue) above each one of these wells/vials 18 which illuminate the sample 12, and thirteen (13) optical fibers 25 below each well/vial 18 collect the sample's signal. The blue LEDs 20_{BL} are used to detect the presence of total coliform, with the image sensor of the camera 50 indirectly measuring the absorption of the transmitted light. The UV LEDs 20_{UV} are used to detect the presence of E. coli by exciting fluorophores in the sample 12. Therefore, when fluorescence is detected in a vial 18, it is classified as containing E. coli. The optical fibers 25 in the bundle of optical fibers 24 are used to map the light passing through the forty (40) wells/vials 18 onto the camera 50, without the use of any mechanical scanning.

The device design is shown in FIGS. 1A, 1B and the system 100 in FIG. 1C. The device 10 uses a 3D-printed structure 14 to hold the components together, and the entire device is placed within an incubator 110 to ensure a constant temperature of 35° C. (other temperatures may be used). A Raspberry Pi microcontroller 40 controls the illumination and a Raspberry Pi camera 50 is used to periodically detect the light from all the forty (40) wells 18. A total of 520 fibers 25 are used, with thirteen (13) collecting light from a given well/vial 18. A plano-convex lens 30 is used below the output of the bundle of optical fibers 24 to help focus the light on the image sensor 52 of the camera 50.

The blue LEDs 20_{BL} are used to detect the colorimetric/absorption signal indicating the presence of total coliform and they operate at a peak wavelength of 400 nm. This gives a strong overlap with o-nitrophenol's absorption spectrum, which is centered at 420 nm. The UV LEDs 20_{UV} used to detect the fluorometric channel operate at a peak wavelength of 365 nm, and are used to excite the 4-MU fluorophores. To eliminate the need for expensive and bulky UV excitation filters, a UV LED 20_{UV} with minimal emission above 400 nm was chosen, which allows the light to be blocked solely by an emission filter 28. Between the LEDs 20_{BL}, 20_{UV} and the glass vials 18 there is a UV-transmitting glass diffuser (not shown), which is used to make the illumination more uniform and reduce the effects of any small movement of the device 10. The LEDs 20 are powered by constant current drivers, which output a current of 20 mA. All of the LEDs 20 are surface mounted to a custom printed circuit board (PCB) 22. To allow for flexibility, the device can either be powered by a rechargeable battery or plugged into a standard outlet.

A 3 mm thick UF-5 Plexiglas sheet is used as a long-pass filter 28, which blocks light below 400 nm, and filters out the light produced by the UV LEDs 20_{UV}. This Plexiglas sheet is an ideal UV filter 28 for this application as it completely blocks the wavelengths desired, does not produce auto-fluorescence, and unlike custom-designed filters, is very cost-effective. This cost-effectiveness is particularly useful as the filter needs to be large (165×110 mm) to cover all the fibers 25.

Once the device 10 has been loaded with the 40 vials 18, the Raspberry Pi microcontroller 40 begins to activate the LEDs 20 and takes an image 90 of the fibers 25 using one wavelength at a time. When the images 90 have been taken, the device 10 waits for 15 minutes with the LEDs 20 off before taking another image 90, which are all saved as raw ‘.mat’ files for processing (other formats may be used). The images 90 using UV excitation have an exposure time of 30 ms while the blue excitation images 90 use an exposure time of 2 ms.

Image Processing

The raw images 90 were processed using MATLAB (The MathWorks, Inc., release R2018a). First, the exact location of the center of each fiber 25 is determined using the first image illuminated by the blue LEDs 20_{BL}. These fiber locations are then associated with the different wells 18 using pre-set manually labeled groupings. To account for minor shifts of the setup, subsequent images 90 are then registered to the initial image 90 using cross correlation.

Following these pre-processing steps, the fiber intensities in each image 90 are measured by summing up the intensities of all the pixels within a 14-pixel radius of the fiber's 25 center. As there is some crosstalk between the different wells 18 in the fluorescence channel, the intensity values for the fibers 15 in each well 18 are reduced according to the intensity of those around them. This normalization is done by multiplying the average intensity of the thirteen (13) fibers 25 under each well or vial 18 by an empirically determined constant and then dividing it by the square of the distance between the wells 18.

The normalized intensities of the fibers 25 in each well 18 are averaged for the final classification. The absorption channel, which is used for the detection of total coliform, is classified as positive when the intensity drops by 5% over 10 successive images 90. The fluorescence channel is classified using a manually-chosen threshold, where the well 18 is marked as positive if the intensity increases more than 20% of the value after the first 75 min, indicating that E. coli is present in the sample 12. In both cases, the first five (5) time points (i.e., the first hour after loading the sample 12 inside the incubator 110) are ignored as there are significant fluctuations in the signal intensities due to the liquid in the vials 18 slowly warming to the temperature of the incubator 110. This causes condensation to form on the sealing film covering the wells 18 over the course of the first hour. The classification threshold for the fluorescence channel was set to be higher than the colorimetric channel as the crosstalk between wells 18 cannot be completely eliminated in the fluorescence detection channel. A visualization of the intensity for the fibers 25 in each well can be seen in FIG. 6C.

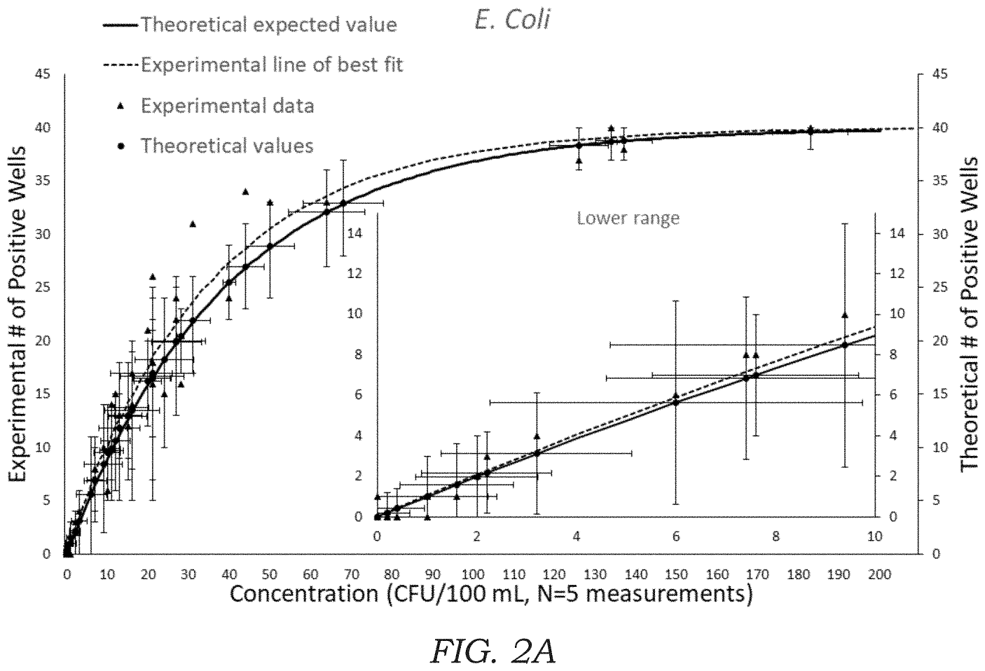

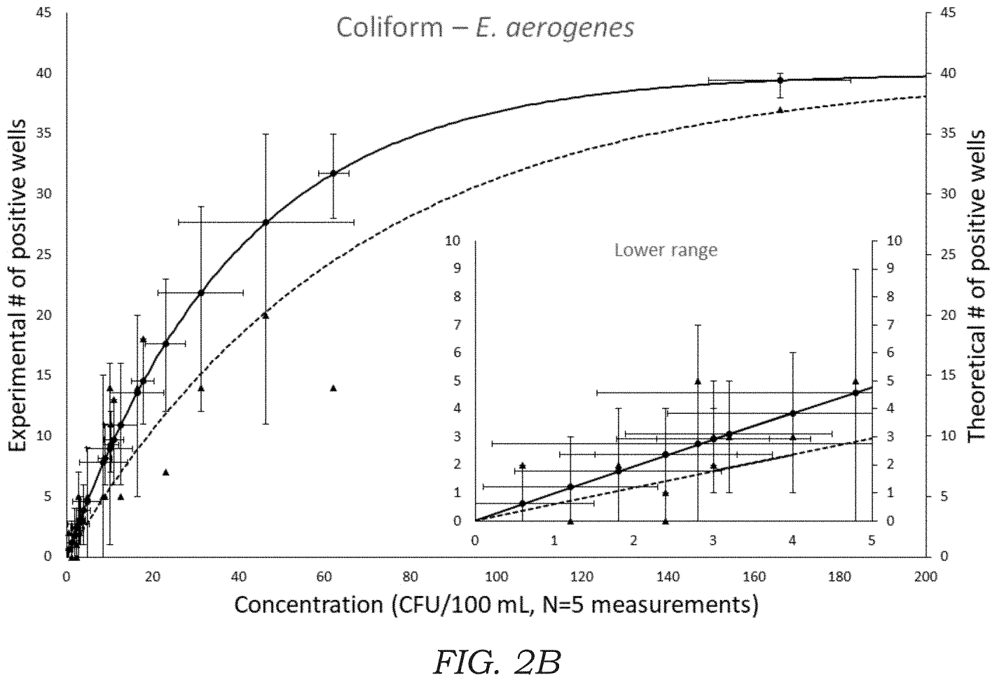

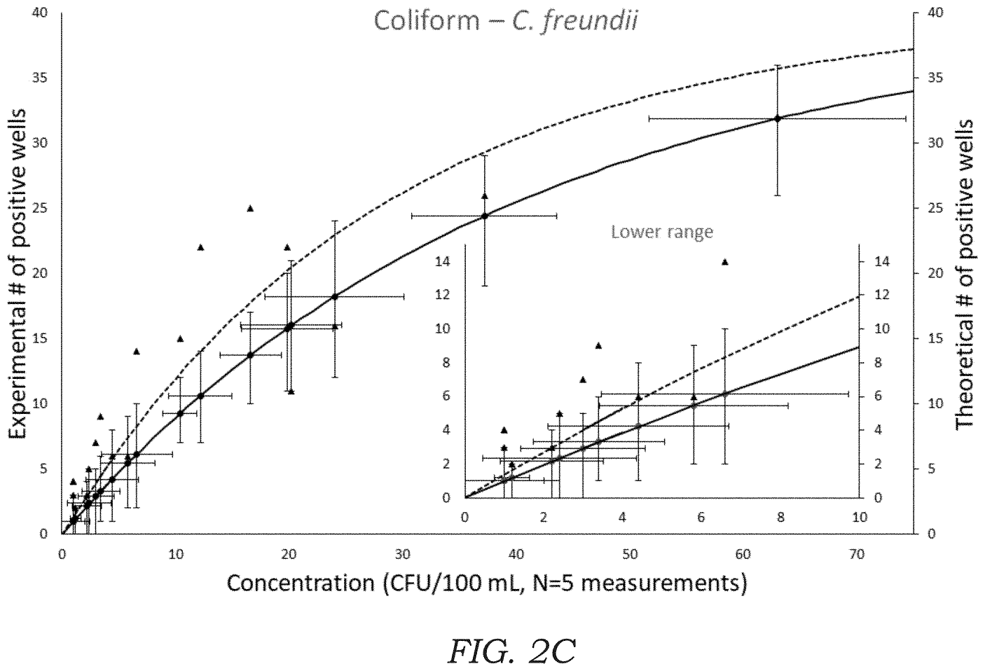

The detection device 10 was validated using E. coli and two different types of total coliform bacteria. Using the procedures described herein, 51 tests were performed using E. coli samples, 27 using E. aerogenes samples, and 19 using C. freundii samples as well as 3 negative samples, in which no bacteria was added, to determine the device 10 performance, sensitivity and limit of detection. FIGS. 2A-2C provides a comparison of the counting efficiency of the device 10 against the gold standard plate count method. In this plot, the number of wells 18 that turned out to be positive is compared with the average of five (5) plate counts. Plate count measurements are required to quantify the concentration of bacteria in the sample tested by the device 10 and constitute the ground truth measurements. The FIGS. also shows the statistically expected number of wells 18 that should be positive for a given plate count measurement. The horizontal error bars for these values are calculated by finding the standard deviation of the plate count (n=5), while the vertical error bars are calculated using the 95% confidence interval of a Monte Carlo simulation, based on the experimental measurement. This simulation was performed by taking the plate count, adding or subtracting a random number of bacteria corresponding to a normal distribution using the measured standard deviation, and finally randomly placing the bacteria into the wells 18 of the 40-well plate.

In FIGS. 2A-2C, the line of best fit is calculated as:

$$Expected number of positive wells = N - N * {( \frac{N - 1}{N} )}^{α * P} \qquad Eq . ( 1 )$$

where N=40 is the number of wells per plate, P is the ground truth bacteria number in the sample (which is the plate count in this case) and α is the constant being fitted for, which represents the efficiency of the device at measuring bacteria concentration compared to the ground truth. When α is equal to one, Eq. (1) gives the theoretical number of positive wells 18 containing bacteria for a given P. For E. coli detection experiments, α was found to be 1.154 (95% confidence between 1.064 and 1.244), which is close to the theoretical detection efficiency. The two total coliform tests had larger deviations from the plate count method, with E. aerogenes having an α of 0.602 (95% confidence between 0.46 and 0.745), and C. freundii having an α of 1.707 (95% confidence between 1.126 and 2.288). These measurements show that there is no consistent trend for either over counting or under counting the bacteria.

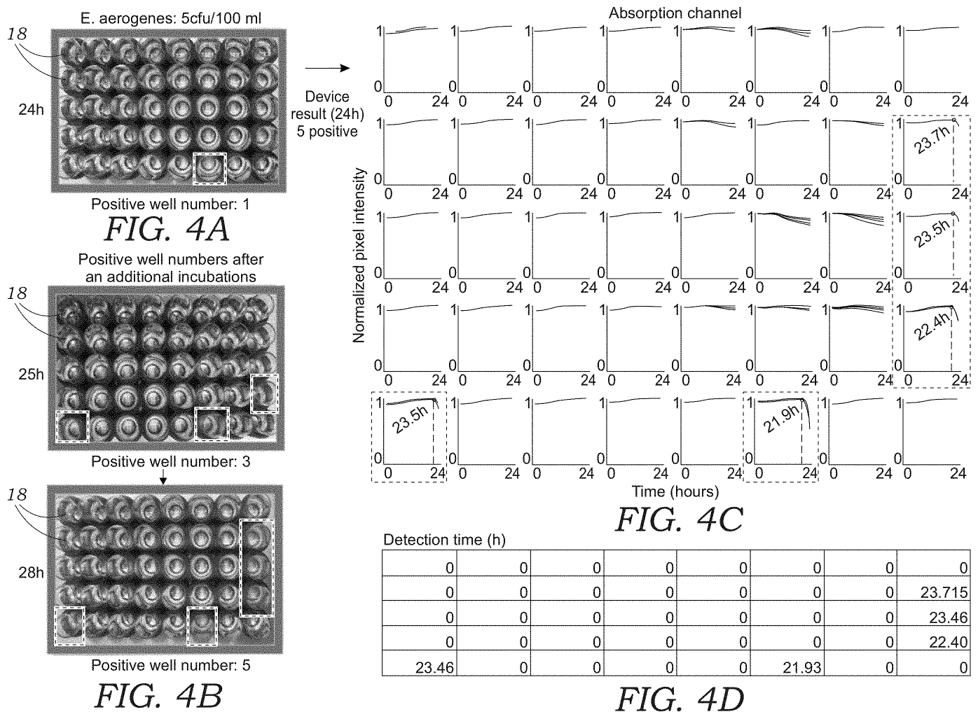

The lower α value of E. aerogenes experiments is likely due to a portion of the bacteria being stressed or injured, and not multiplying efficiently for the test to report positive within the 24-hour window. It is important to note that for the undercounted E. aerogenes measurements, the device 10 was able to obtain better results than a standard visual count of the positive wells since the automated detection is more sensitive than the human eye. One example of this can be seen in FIGS. 4C and 4D. For all other bacteria measurements, the visual positive well counts and the device counts were the same.

The overcounting of the C. freundii samples compared to the plate counts, with an α of 1.707, can potentially be due to the bacteria being in the viable but non-culturable (VBNC) state. Bacteria can enter the VBNC state for reasons such as environmental stress. When this happens, they can preserve some metabolic activity (detected by the device 10) but lose their ability to grow on an agar plate. Therefore, the concentration of bacteria detected by enzyme-based methods, such as the presented device 10, can be higher than the concentration detected using culture-based methods.

If the number of bacteria detected by the device needs to be quantified, Eq. 1 can be rearranged to provide a probabilistic estimation:

$$Estimated number of bacteria = \frac{log ( N - W )}{log ( \frac{N - 1}{N} )} \qquad Eq . ( 2 )$$

where W is the number of positive wells counted by the device. At low concentrations the number of bacteria tested by the sample can be accurately estimated using Eq. (2). However, at higher concentrations as the probability of multiple bacteria to end up at the same well increases, Eq. (2) will undercount bacteria.

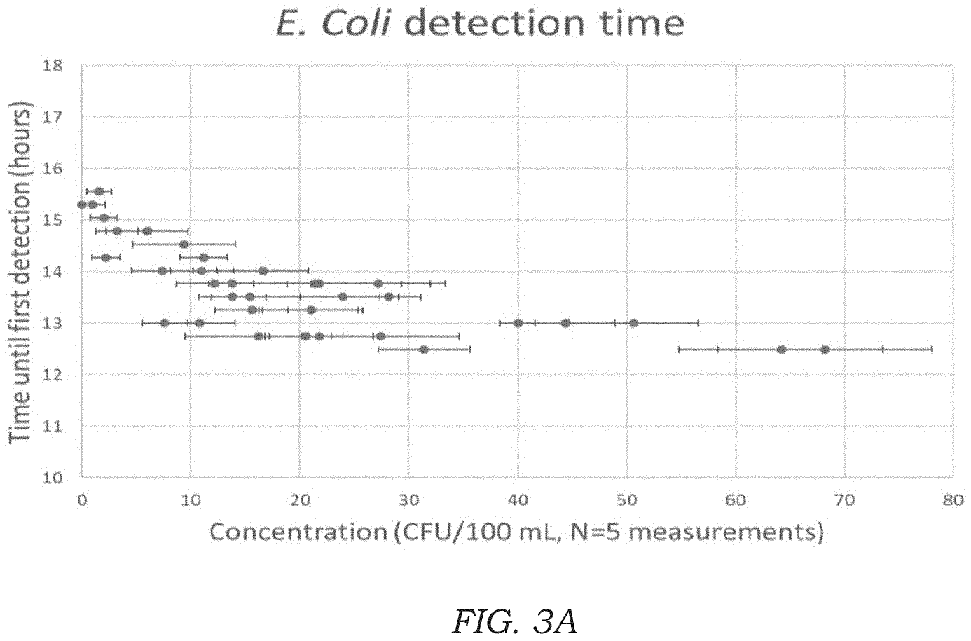

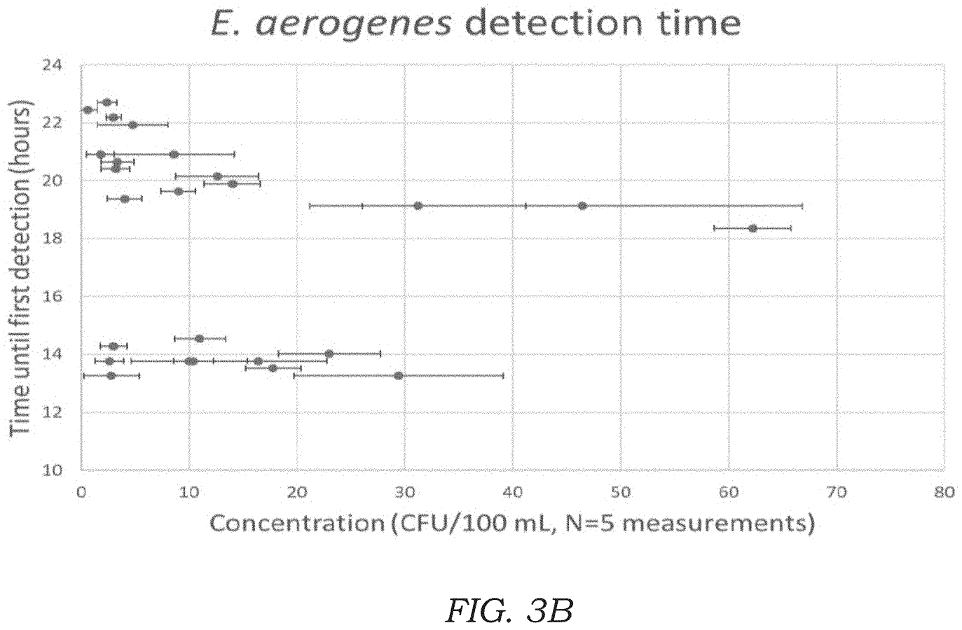

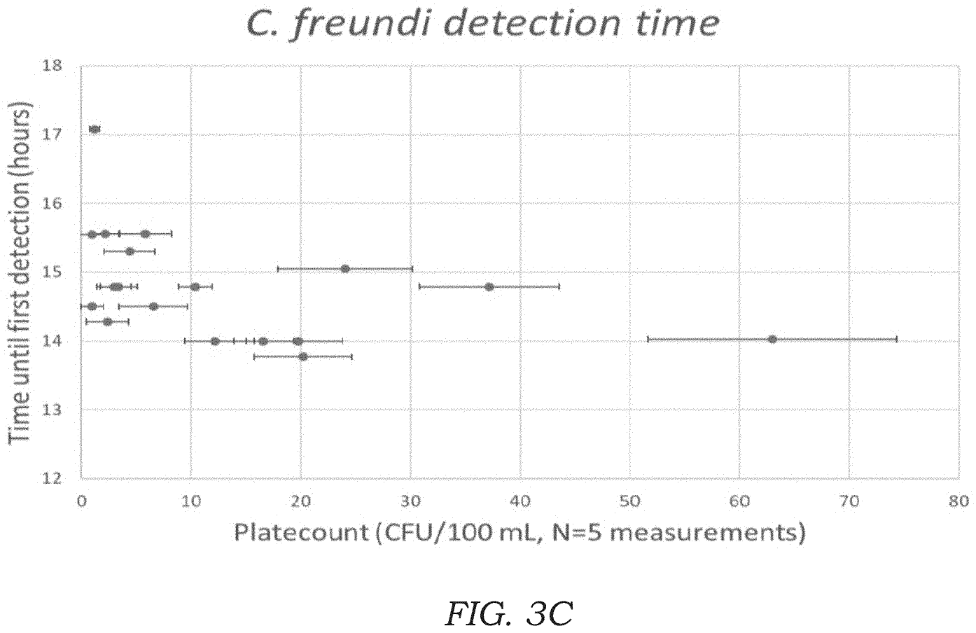

FIGS. 3A-3C demonstrate one of the important benefits of the presented device 10: it can automatically detect bacteria several hours faster than the standard Colilert® method. While the exact detection time depends on several factors, FIGS. 3A-3C shows that the device 10 is capable of bacteria detection in <16 hours, i.e., 8 hours faster than manual inspection. This is even the case at lower bacteria concentrations: for water samples 12 with a concentration of ≤5 CFU/100 mL, the device 10 was able to detect the presence of bacteria on average within 15.0 and 15.1 hours of the start of the incubation period for E. coli and C. freundii, respectively. The exception to this trend is a portion of the E. aerogenes samples, which, as discussed earlier, take significantly longer to be detected, e.g., 21.4 hours after the start of the incubation for the same concentration range. It should be noted that for these E. aerogenes samples 12 it also took significantly longer than 24 hours to exhibit color change or fluorescence signal using the standard Colilert® method, which indicates potentially damaged or stressed bacteria. See for example FIGS. 4A-4D, which reports that the visual signal change, indicative of the presence of the bacteria, occurred only after 28 hours for some of the vials in this E. aerogenes sample, whereas for the same vials the device 10 detected the presence of the bacteria several hours earlier compared to the visual inspection.

This significant decrease in the detection time when compared to the traditional Colilert® method is a result of two factors. First, since the device 10 is completely automated, it performs measurements at regular intervals rather than waiting for the full 24 hours. Additionally, since it performs comparative quantitative analysis, the device 10 can make more sensitive measurements than is possible with a manual end-point qualitative measurement using the Colilert® method. FIGS. 4C and 4D show a demonstration of this increased sensitivity, showing that the device 10 can detect all of the five (5) positive wells 18 under 24 hours while a visual inspection at 24 hours is unable to detect any color changes that indicate the growth of the E. aerogenes. Only after the same samples 12 have been incubated for an additional four hours, visual inspection was then able to determine that all of these wells 18 are indeed positive.

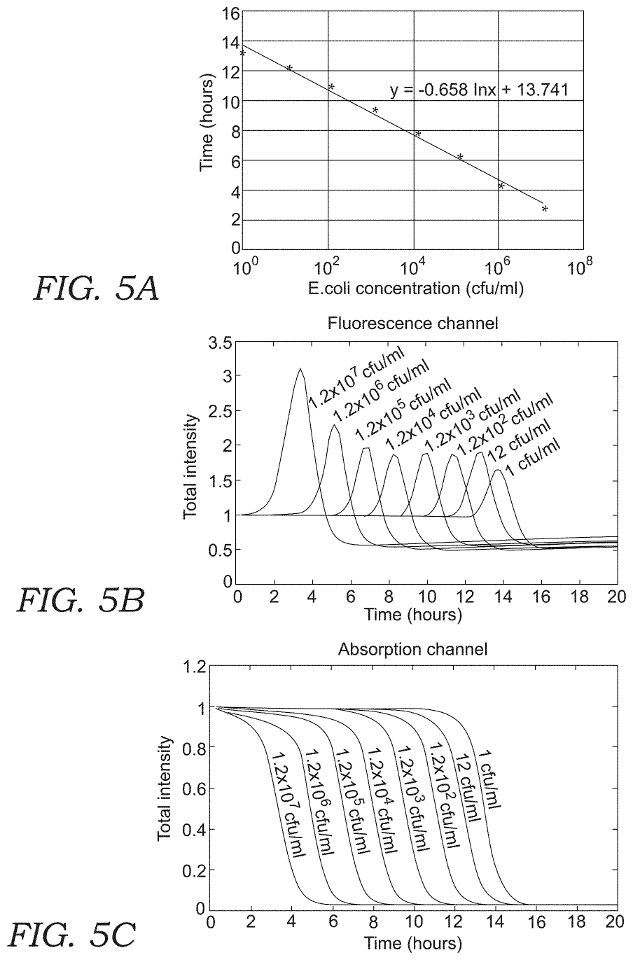

FIGS. 5A-5C also shows that as the bacteria concentration increases, the detection time further decreases as a larger number of bacteria can interact with the enzymes at an earlier time point. This point is illustrated in FIG. 5A, which reports the reduction in the detection time as the concentration of bacteria is raised to higher levels. Using the average of five (5) measured wells 18 at each concentration, the detection time is found to get ˜0.66 hours less for each order of magnitude increase in the concentration of bacteria over the range of 1 to 1.2×10⁷ CFU/mL. For example, at a concentration of 1 CFU/mL, the device 10 takes an average of 13.7 hours to automatically detect the presence of the bacteria, while at a concentration of 1.2×10⁷ CFU/mL it only takes 2.8 hours. FIGS. 5B and 5C demonstrate how the intensity measured by the device 10 changes over time for the fluorescence and absorption channels of the device, respectively.

As the device 10 is designed to test drinking water, all the testing has been performed using non-turbid water. EPA regulations require the turbidity in drinking water to be below 1 nephelometric turbidity unit (NTU) when direct or conventional filtration is used, and below 5 NTU otherwise. At a turbidity level of 5 NTU, less than 7% of the light passing through the sample will be absorbed. Since the device 10 makes a determination of bacterial growth according to the relative changes in the signal intensity (as a function of time), any absorption from these low levels of turbidity that drinking water might exhibit will therefore not impact the operation of the device 10.

The device 10 including the housing 14 and may be 3D-printed that, in one embodiment, is controlled by a Raspberry Pi microcontroller 40 which can perform automated detection of both E. coli and total coliform in a 100 mL water sample 12 using the Colilert® reagent. The device 10 is more sensitive than a manual count, and that, because of this increased sensitivity, it can automatically detect the presence of bacteria faster than it is possible through a manual measurement. The device 10 is able to perform these measurements according to the EPA standard, which requires testing of 100 mL of water sample, and it can identify a single organism in this 100 mL sample (i.e., 1 CFU/100 mL). Additionally, it is capable of performing this automated detection within less than 16 hours. This time can be further reduced as the bacteria concentration is increased.

The automatic classification of the wells 18 as positive (+) or negative (−) eliminates the need for a trained operator as well as the risk of a counting error. Additionally, since no specialized skills are required for its operation, it can be used with minimal training. This simplicity also allows the sample preparation to be performed in only a few minutes, while still being effective, giving a definitive result in less than 24 hours.

By using a Raspberry Pi microcontroller 40 and camera 50 with a CMOS image sensor 52 to perform the detection, optical fibers 25 to collect the light, Plexiglas as an inexpensive fluorescence emission filter 28, and UV LEDs 20_{UV} for illumination (without the need for an excitation filter), the blue LEDs 20_{BL}, the device 10 is rather cost-effective, with its parts costing ˜$600 under low volume manufacturing, which can be significantly reduced with economies of scale. Therefore, it is applicable in a variety of settings, particularly in areas where access to a central lab or transportation of the sample are not feasible. In the future, the device 10 can be modified to use a custom incubator 110, which would allow the device to be field-portable and even more cost-effective to use. For example, the system 100 may be sold with the device 10 and incubator 110 together. Reagent(s) may also be provided as part of a kit.

While embodiments of the present invention have been shown and described, various modifications may be made without departing from the scope of the present invention. For example, while the testing reagent comprises a first substrate (o-nitrophenyl-β-D-galactopyranoside (ONPG)) and a second substrate (4-methylumbelliferyl-β-D-glucuronide (MUG)) it should be appreciated that other reagent(s) may be used to test for bacteria. The invention, therefore, should not be limited, except to the following claims, and their equivalents.

1. A system for detecting the presence of E. coli and total coliform in a water sample comprising:
a sample holder configured to hold a plurality of smaller volumes of the sample and a testing reagent therein;
a plurality of light sources disposed above the plurality of smaller volumes of the sample, wherein each of the plurality of smaller volumes of the sample within the sample holder is illuminated with first and second light sources of the plurality of light sources emitting light at different wavelengths;
a bundle of optical fibers having an input end and an output end, the input end of the bundle comprising a plurality of optical fibers located adjacent to the plurality of smaller volumes of the sample and configured to receive light passing through the plurality of smaller volumes of the sample from the first and second light sources;
a lens configured to receive light emitted from the output end of the bundle of optical fibers;
a camera configured to capture images of the light passing through the lens from the bundle of optical fibers; and
image processing software configured to calculate a light intensity in a first wavelength channel and a second wavelength channel at different times, wherein the image processing software outputs a positive/negative indication for E. coli and total coliform for the water sample.

2. The system of claim 1, further comprising a long-pass filter interposed between the sample holder and the input end of the bundle of optical fibers.

3. The system of claim 1, wherein the system is portable.

4. The system of claim 1, wherein the system measures light intensity in the first and second channels a plurality of times per hour.

5. The system of claim 1, further comprising a housing containing the sample holder, plurality of light sources, bundle of optical fibers, lens, and camera.

6. The system of claim 5, wherein the housing is disposed in an incubator.

7. The system of claim 1, further comprising a microcontroller and/or one or more microprocessors configured to periodically illuminate the plurality of smaller volumes with the plurality of light sources and obtain images of light passing through the plurality of smaller volumes transmitted by the bundle of optical fibers.

8. The system of claim 1, wherein the positive/negative indication for E. coli for any particular sample volume is based on based on a threshold increase in observed intensity over time.

9. The system of claim 8, wherein the threshold increase comprises at least a 20% increase.

10. The system of claim 1, wherein the positive/negative indication for total coliform for any particular sample volume is based on based on a threshold decrease in observed intensity over time.

11. The system of claim 10, wherein the threshold increase comprises at least a 5% decrease.

12. The system of claim 1, wherein the testing reagent comprises a first substrate (o-nitrophenyl-β-D-galactopyranoside (ONPG)) and a second substrate (4-methylumbelliferyl-β-D-glucuronide (MUG)).

13. The system of claim 1, wherein the image processing software further is configured to output a concentration or concentration range of E. coli and total coliform in the water sample.

14. A method for detecting the presence of E. coli and total coliform in a water sample comprising:
mixing the water sample and a testing reagent;
dividing the mixture into a plurality of smaller volumes of the water sample and the testing reagent;
loading the plurality of smaller volumes of the water sample and the testing reagent into the sample holder of the device of claim 1;
periodically illuminating the plurality of smaller volumes of the water sample and the testing reagent with light from the first light source and capturing images of the light passing through the lens from the bundle of optical fibers associated with the plurality of smaller volumes of the water sample;
periodically illuminating the plurality of smaller volumes of the water sample and the testing reagent with light from the second light source and capturing images of the light passing through the lens from the bundle of optical fibers associated with the plurality of smaller volumes of the water sample;
processing the periodically obtained images obtained with the first light source and the second light source with image processing software configured to calculate a light intensity in first and second channels at the periodic times, wherein the image processing software outputs a positive/negative indication for E. coli and total coliform for the water sample based on a change in light intensity over time.

15. The method of claim 14, wherein the positive/negative indication for E. coli is based on based on a threshold increase in observed intensity over time.

16. The method of claim 15, wherein the threshold increase comprises at least a 20% increase.

17. The method of claim 14, wherein the positive/negative indication for total coliform is based on based on a threshold decrease in observed intensity over time.

18. The method of claim 17, wherein the threshold increase comprises at least a 5% decrease.

19. The method of claim 14, wherein the image processing software further is configured to output a concentration or concentration range of E. coli and total coliform in the water sample.
